(19) United States
(12) Patent Application Publication
(10) Pub. No.: US 2004/0259269 A1
(43) Pub. Date: Dec. 23, 2004

(54) Method for detection of molecular species in a crude sample using capillary electrophoresis

(75) Inventors: Shin-Fuw Lin, Lexington, MA (US); George E. Barringer JR., West Groton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Groton Biosystems, Acton, MA

(21) Appl. No.: 10/601,096

(22) Filed: Jun. 20, 2003

(51) Int. Cl. G01N 33/561

(52) U.S. Cl. 436/516

(57) ABSTRACT

A method for detecting at least one molecular species in a crude sample is disclosed. The method comprises: acquiring a crude sample containing at least one molecular species; separating from the molecular species sample components that have size and masses greater and/or smaller than the molecular species of interest; pre-focusing molecular species in the crude sample within the capillary electrophoresis apparatus one or more times by applying voltage to the capillary for a period of time sufficient to cause electrophoretic migration of at least one molecular species in the sample up to a boundary between a low-electrolyte plug and a high-electrolyte buffer and applying a negative pressure differential to the capillary for a period of time sufficient to cause the pre-focused molecular species to be pushed substantially to its original position; electrophoretically separating the molecular species in the crude sample; and detecting the separated molecular species.

### BACKGROUND OF THE INVENTION

Electrophoretic separation of biopolymers and small molecules is one of the most widely used separation technique employed by modem biology and biotechnology. Molecular species, or analytes, such as peptides, proteins, and oligonucleotides are separated by causing them to migrate at different rates in a separation medium under the influence of an electric field. The separation medium can be a buffer solution, or a low to moderate concentration of an appropriate gelling agent such as agarose or polyacrylamide. When gel separation medium is used, separation of analytes is partly based on their molecular sizes as the analytes are sieved by the gel matrix. Given equal electric charge density, smaller molecules move relatively more quickly than larger ones through a gel of a given pore size, depending in part on the concentration of the polymer in the gel.

A particularly preferred electrophoresis format is capillary electrophoresis (CE), where the electrophoresis is performed in a capillary tube having a small internal diameter. Capillary electrophoresis results in enhanced separation performance over traditional slab-based formats because the superior ability of the narrow-bore capillary to dissipate heat allows for high electrical fields to be employed thereby resulting in fast separations while sample diffusion is minimized.

While capillary electrophoresis is a very powerful and versatile technology used to analyze, i.e., to separate and detect components of complex mixtures of materials, it is ultimately influenced by delicate variations in acidity (pH), ionic strength, temperature, viscosity and other physical characteristics of the mixture. Intrinsic properties of the analytes being studied can cause variations. Quick detection of analytes in a crude sample is especially challenging. The time from sample extraction from a complex bioreactor mixture to analysis of a molecular species of interest can easily be several hours. Such a slow analysis time leads to poor optimization of reactor processes, resulting in lowered yields, increased costs, increased purification demands, and increased amounts of potentially hazardous biological waste.

Accordingly, new rapid methods of detecting analytes in a crude sample using CE are sought that would remove excess ions, maintain constant pH and viscosity, and be amenable to automation.

### SUMMARY OF THE INVENTION

The present invention is a method and an apparatus for detecting molecular species in a crude sample using capillary electrophoresis.

In one embodiment, the present invention is a method of preparing a crude sample for detecting at least one molecular species of interest, comprising: (a) acquiring a crude sample containing at least one molecular species of interest, one or more rough components that are larger than the molecular species of interest, and one or more fine components that are smaller than the molecular species of interest; (b) separating from the molecular species of interest at least a portion of the rough component and, optionally, at least a portion of the fine component of the crude sample, thereby producing a cleared sample comprising the at least one molecular species of interest; (c) optionally, at least partially denaturing the at least one molecular species of interest contained in a cleared sample; and (d) pre-focusing the at least one molecular species of interest in the cleared sample, wherein steps (a) through (d) are performed for a number of times sufficient to bring the concentration of at least one molecular species of interest in the sample up to the level of detection.

In another embodiment, the present invention is a method of detecting at least one molecular species of interest, comprising: (a) acquiring a crude sample containing at least one molecular species of interest, one or more rough components that are larger than the molecular species of interest, and one or more fine components that are smaller than the molecular species of interest; (b) separating from the molecular species of interest at least a portion of a rough component and, optionally, at least a portion of the fine component of the crude sample, thereby producing a cleared sample comprising the at least one molecular species of interest; (c) optionally, at least partially denaturing the at least one molecular species of interest contained in a cleared sample; (d) pre-focusing the at least one molecular species of interest in the cleared sample; (e) electrophoretically separating the molecular species of interest in the cleared sample and (f) detecting the separated molecular species of interest. Preferably, steps (a) through (d) are performed for a number of times sufficient to bring the concentration of the at least one molecular species of interest in the sample up to the level of detection.

In another embodiment, the present invention is a method of detecting at least one molecular species of interest, comprising: (a) acquiring a crude sample containing at least one molecular species of interest, one or more rough components that are larger than the molecular species of interest, and one or more fine components that are smaller than the molecular species of interest; (b) separating from the molecular species of interest at least a portion of a rough component and, optionally, at least a portion of the fine component of the crude sample, thereby producing a cleared sample comprising the at least one molecular species of interest; (c) introducing the cleared sample into a separation device, said device comprising (i) a capillary, at least partially filled with a high-electrolyte buffer, said capillary having an inlet end and an outlet end, (ii) a means for applying voltage between the inlet end and the outlet end of said capillary, and (iii) a means for applying pressure differential between the inlet end and the outlet end of said capillary; (d) pre-focusing the at least one molecular species of interest in the cleared sample; (e) electrophoretically separating the molecular species of interest in the cleared sample; and (f) detecting the separated molecular species of interest. Preferably, steps (a) through (d) are performed for a number of times sufficient to bring the concentration of the at least one molecular species of interest in the sample up to the level of detection.

In another embodiment, the present invention is a method of detecting at least one molecular species of interest in a sample, comprising: (a) acquiring a crude sample containing at least one molecular species of interest, at least one rough component, larger than the molecular species of interest, and at least one fine component smaller than the molecular species of interest; (b) separating the rough component and, optionally, the fine component from the molecular species of interest, thereby producing a cleared sample, thereby producing a cleared sample comprising the at least one molecular species of interest; (c) optionally, adjusting the acidity of the cleared sample; (d) optionally, at least partially denaturing the at least one molecular species of interest in the cleared sample; (e) optionally, removing insoluble contaminants from the cleared sample; (f) introducing the cleared sample into a separation device, said device comprising (i) a capillary, at least partially filled with a high-electrolyte buffer, said capillary having an inlet end and an outlet end, (ii) a means for applying voltage between the inlet end and the outlet end of said capillary and (iii) a means for applying pressure differential between the inlet end and the outlet end of said capillary; (g) pre-focusing the at least one molecular species of interest in the cleared sample; (h) electrophoretically separating the molecular species of interest in the cleared sample; and (i) detecting the separated molecular species of interest. Preferably, steps (a) through (g) are performed for a number of times sufficient to bring the concentration of the at least one molecular species of interest in the sample up to the level of detection.

In another embodiment, the present invention is a method of detecting at least one protein species of interest in a crude sample, comprising: (a) acquiring a crude sample containing at least one protein species of interest, at least one rough component, larger than the at least one protein species of interest, and at least one fine component smaller than the at least one protein species of interest; (b) separating the rough component and, optionally, the fine component from the protein species of interest, thereby producing a cleared sample comprising at least one protein species of interest; (c) optionally, adjusting the acidity of the cleared sample to between about 5.0 and about 7.0 pH units; (d) optionally, at least partially denaturing the at least one protein species in the cleared sample by (i) adding at least one detergent to the cleared sample and (ii) heating the cleared sample in the presence of at least one detergent; (e) optionally, removing insoluble contaminants contained in the cleared sample; (f) introducing the cleared sample into a separation device whereby a low-electrolyte aqueous solution plug having the acidity of about 7.0 pH units is introduced into the separation device, said low-electrolyte aqueous plug disposed between the high-electrolyte buffer and the cleared sample at the inlet end the capillary, thereby forming a boundary between the low-electrolyte aqueous plug and the high-electrolyte buffer; (g) pre-focusing and, optionally, desalting the cleared sample; (h) electrophoretically separating the protein species in the cleared sample, comprising the step of applying voltage between the inlet end and the outlet end of the capillary; and (i) detecting the separated protein species. The device preferably comprises (i) a capillary, at least partially filled with a high-electrolyte buffer, having an acidity between about 7.0 and about 9.0 pH units, said capillary having an inlet end and an outlet end, (ii) a means for applying voltage between the inlet end and the outlet end of said capillary and (iii) a means for applying pressure differential between the inlet end and the outlet end of said capillary. In a preferred embodiment, said re-focusing and desalting comprises (i) applying voltage between the inlet end and the outlet end of the capillary for a period of time sufficient to cause electrophoretic migration of the at least one protein species in the cleared sample up to the boundary between the low-electrolyte aqueous plug and the high-electrolyte buffer, thereby pre-focusing and desalting the cleared sample and (ii) applying a pressure differential between the inlet end and the outlet end of the capillary for a period of time sufficient to cause the pre-focused protein species of interest to be pushed substantially toward the inlet end of the capillary.

The benefits of repeated pre-focusing and sample displacement include sample concentration with concomitant desalting of the sample, which in turn, lead to increased reliability and reproducibility of the analytical results and higher method sensitivity and resolution. Accordingly, the method of the present invention allows for detection of molecular species of interest present at very low concentrations. The method of the present invention can further be used for high-speed on-line analysis and monitoring of crude reaction products extracted directly from a reaction vessel. The method of the present invention is amenable to automation and, therefore, can be used for high throughput applications. The combination of high throughput with provision of automatic control allows the preparation and analysis of molecular species at a higher rate, enabling higher frequency of sampling. Improved sampling frequency permits analysis, control and optimization of time-sensitive, production scale bioreactor processes, which, in turn, lead to higher yields, reduced costs, lower purification demands, and decreased amounts of potentially hazardous biological waste.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIG. 1 is a schematic of a separation device suitable for practicing the method of the present invention.

FIG. 2A to 2D show schematic representation of one embodiment of sample pre-focusing. FIG. 2A shows the position of the sample, low-electrolyte plug and high-electrolyte buffer. FIG. 2B schematically depicts positions of charged ionic species during pre-focusing. FIG. 2C schematically depicts positions of charged ionic species at the end of pre-focusing before the application of the negative pressure. FIG. 2D schematically depicts positions of charged ionic species at the end of pre-focusing after the application of the negative pressure.

FIG. 3 depicts a stationary capillary electrophoresis circuit that can be controlled to conduct the steps the method of the present invention.

FIG. 4 depicts a more detailed schematic of the capillary electrophoresis circuit.

FIG. 5 is a comparison electrophoregram of a sample aliquot spiked with molecular weight markers prepared with (bold trace) and without (thin trace) the step of sample stacking.

FIG. 6 is an electrophoregram showing the effects of sample stacking using the method described herein. The bottom curve corresponds to the unstacked molecular marker profile. The top curve corresponds to 2.5 fold overloaded markers. Notice the peak broadening. The middle curve corresponds to 2 fold overloaded stacked sample of molecular weight standards.

FIG. 7 is an electrophoregram showing the detection of the protein of interest, muPGF in timed aliquots. The topmost curve corresponds to an aliquot of batch B1 sampled at 16.5 hours overlayed by a sample of purified protein for reference. The second curve from the top corresponds to a 16.5-hour aliquot of batch B1 without an overlay. The second curve from the bottom corresponds to an aliquot of batch B2 sampled at 8.75 hours. The bottommost curve corresponds to a 16.5-hour aliquot of batch B2.

### DETAILED DESCRIPTION OF THE INVENTION

While this invention has been particularly shown and described with references to preferred embodiments thereof, it will be understood by those skilled in the art that various changes in form and details may be made therein without departing from the scope of the invention encompassed by the appended claims.

Unless stated otherwise, the following terms and phrases as used herein are intended to have the below-defined meanings.

The term “electrophoresis” as used herein refers to a method of separation of charged species including ions, molecules, etc., based on their migration rate in an electric field.

The term “capillary” as used herein refers to a tube or channel or other structure capable of supporting a volume of separation medium for carrying out electrophoresis. The geometry of a capillary may vary widely and includes tubes with circular, rectangular or square cross-sections, channels, groves, plates, and the like, and may be fabricated by a wide range of technologies. An important feature of a capillary for use with the invention is the surface-to-volume ratio of the surface in contact with the volume of separation medium. High values of this ratio permit better heat transfer from the separation medium during electrophoresis. Preferably, values in the range of about 0.4 to about 0.04 are employed. These correspond to the surface-to-volume ratios of tubular capillaries with circular cross-sections having inside diameters in the range of about 10 μm to about 100 μm.

The term “separation medium” refers to a medium in which an electrophoretic separation of sample components takes place. Separation media typically comprise several components, at least one of which is a charge-carrying component, or electrolyte. The charge-carrying component is usually part of a buffer system for maintaining the separation medium at a defined pH. Media used for separating polynucleotides, proteins or other biomolecules that have different sizes, but identical charge-frictional drag ratios in free solution, can further include a sieving component. Such sieving component is typically composed of a cross-linked polymer gel, e.g., cross-linked polyacrylamide or agarose, or a polymer solution, e.g., a solution of polyacrylamide, hydroxyethyl cellulose, and the like.

The term “crude” refers to a sample taken directly from a reaction vessel or a cell culture. The crude sample is preferably liquid, typically aqueous, mixture that can contain one or more molecular species to be detected, typically macromolecules, and can also contain fine components, e.g., salts, nutrients, metabolites, and the like molecules that are smaller than the molecular species to be detected; and rough components, e.g., cells, cell fragments, particulate contaminants, insoluble contaminants, and other molecules larger than the macromolecule, and the like.

As used herein, rough components can include soluble and insoluble components. Insoluble components include cells, fragments of cells, non-cellular tissue fragments, insoluble agglomerations of macromolecules, particulate contaminants, and the like. Soluble rough components include smaller fragments of cells, macromolecules that are larger than the macromolecule or are greater in molecular weight than the molecular weight of the macromolecule, and the like.

As used herein, fine components include soluble components. Soluble fine components include macromolecules that are smaller than the macromolecule or are lesser in molecular weight than the molecular weight of the macromolecule. Also included are small organic and inorganic molecules, for example, salts, amino acids, nucleic acids, cofactors, nutrients, metabolites, other macromolecules, fragments of the macromolecule, other biomolecules, and the like.

As used herein, salt components include salts formed from cations such as sodium, potassium, lithium, cesium, magnesium, manganese, copper, zinc, calcium, iron, ammonium, alkylammonium, phosphonium, sulfonium, and the like. Salt components also include anions including halides, sulfates, thiosulfates, sulfonates, sulfites, nitrates, nitrites, carboxylates, phosphates, phosphates, phosphonates, carbonates, hydroxides, and the like.

The liquid in the liquid mixture containing the macromolecule can be any solvent, for example, water, organic solvents such as alcohols, e.g., methanol, ethanol, isopropanol, t-butanol, and the like; ethers, e.g., dimethyl ether, diethyl ether, tetrahydrofuran, and the like; ketones, e.g., acetone, methyl ethyl ketone, and the like; aromatic solvents, e.g., benzene, toluene, and the like; halogenated solvents, e.g., chloroform, carbon tetrachloride, trichloroethylene, and the like; polar aprotic solvents, e.g., dimethyl sulfoxide, nitrobenzene, dimethyl formamide, n-methylpyrrolidone, acetonitrile, and the like; mixtures thereof, and the like. Typically, the liquid can be water, optionally with small amounts of one or more organic solvents that are miscible with water, e.g., ethanol, isopropanol, acetonitrile, and the like.

As used herein, a “buffer” can be any liquid that can be added to the mixture to maintain or change the concentration of a particular component, or to combine an additive to change the properties of the process. For example, an ionic buffer, e.g., a pH buffer, can change or maintain the pH of the liquid mixture; a denaturation buffer can contain a denaturation agent; a desalination buffer can be a liquid substantially free of salts or substantially free of a particular salt, e.g., sodium chloride; a lysis buffer can be a liquid that contains a lysing agent (e.g., a detergent) or can be sufficiently low in ionic strength to lyse cells by ionic shock; and the like. Lysing agents can include enzymes, e.g., L-lysine decarboxylase, lysostaphin, lysozyme, lyticase, mutanolysin, and the like. Lysing agents can include detergents, e.g. glycocholic acid sodium salt hydrate, lithium dodecyl sulfate, sodium cholate hydrate, sodium dodecyl sulfate, hexadecyltrimethylammonium bromide, N-nonanoyl-N-methylglucamine, octyl-b-D-1-thioglucopyranoside, 3-(N,N-dimethyloctadecylammonio)propanesulfonate, and the like.

“Protein”, “polypeptide” and “peptide” may be used interchangeably herein when referring to a naturally occurring or recombinant gene product, e.g., as may be encoded by a coding sequence. A “polypeptide” or “peptide” also may refer to a polymer of amino acids, either naturally occurring or synthetically produced. A “peptide nucleic acid” or “PNA” refers to an analogue of a nucleic acid in which the backbone of the molecule is not sugar-phosphate, but rather a peptide or peptidomimetic. A detailed description of PNAs may be found in Nielsen, et al., Curr. Issues Mol. Biol. (1999) 1:89-104. The term “peptidomimetic” refers to a molecule containing peptide-like structural elements that is capable of mimicking the biological action (s) of a natural parent polypeptide. “Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. The term “aptamers’ refers to fragments of single stranded DNA or RNA bind target molecules and that are usually less than 100 nucleotides in length. Aptamers are the nucleic acid equivalent of antibodies. (See, e.g., Patel D. J. et al., “Structure, Recognition and Adaptive Binding in RNA Aptamer Complexes”, J. Mol. Biol. (1997) 272:645-664).

In one embodiment, the present invention is a method of detecting at least one molecular species of interest in a crude sample. Molecular species that can be detected by the method of the present invention can be selected from the group consisting of antibodies, proteins, peptides, peptidomemetics, peptide-nucleic acids, oligonucleotides, aptamers, lipids, polysaccharides, liposaccharides, lipoproteins, glycoproteins, and small molecules. Preferably, the molecular species are protein or peptides. In a preferred embodiment, the sample is taken directly from a reaction vessel such as a fermentation tank or a bioreactor. The reaction vessel can contain live microorganisms. In one embodiment, molecular species are secreted by microorganisms. In another embodiment, the molecular species are produced by microorganisms in the form of inclusion bodies. As used herein, the term “inclusion bodies” refers to insoluble aggregates of misfolded protein that is a result of a gene product overproduction in prokaryotic microorganisms such as bacteria, e.g., E. coli. If the molecular species are produced by microorganisms in the form of inclusion bodies, the method of the present invention can include a lysis step wherein at least a portion of the cells are lysed to release the molecular species. Lysing can be conducted using any method of lysing cells well-know to the art, for example, heating, sonic disruption, addition of lysing agents, e.g., detergents, changes in ionic strength, e.g., by dilution with water or combination with a lysis buffer, and the like. Lysis of the cells can be followed by the step of refolding whereby the released molecular species is allowed to at least partially assume its native 3-dimensional conformation. Refolding is carried out according to any of the protocols known in the art and typically comprises dilution of the solution containing the molecular species of interest in the presence of salts, detergents and reducing agents such as glutathion, dithiothreitol and cystein.

In one embodiment, the present invention is a method of preparing a crude sample for detecting at least one molecular species of interest. The crude sample is acquired from a reaction mixture, preferably from a bioreactor. The crude sample preferably contains at least one molecular species, one or more rough components that are larger than the molecular species, and one or more fine components that are smaller than the molecular species. At least a portion of the rough component and, optionally, at least a portion of the fine component of the crude sample are separated from the molecular species using any of the techniques known in the art for separating molecular species based on size and/or mass and can comprise filtering and/or centrifuging.

In one embodiment, centrifuging can be used to remove larger particles such as cellular debris and insoluble contaminants. Any of the commercially available centrifuges can be used, for example Bio-Rad® Minicentrifuge Model SD™. Preferably, centrifugation is performed at about 7000 g for about 1 minute. The volume of the crude sample necessary for preparing for introduction into a separating device can be determine by a skilled artisan based on the expected concentration of a molecular species to be detected.

In another embodiment, an apparatus described in co-pending patent applications filed under the Ser. Nos. ______ attorney docket number 3555.1002-000 and 3555.1003-000 can be used to separate at least portions of the rough and fine components from the molecular species of interest. The entire teachings of both applications are herein incorporated by reference.

To accomplish the separation of the components, the acquired crude sample is directed through a filter suitable for removing at least a portion of components that are larger than the molecular species, i.e., greater in diameter or molecular weight than the molecular species. Such filter is hereinafter referred to as “rough filter”. A pressure differential across a rough filter directs at least a portion of the liquid, the molecular species, and the fine components through the filter, separating at least a portion of rough components at the rough filter. Preferably, the rough filter removes components that are greater in molecular weight than the molecular weight of the macromolecule by about 150%, more preferably about 125%, even more preferably about 110%, and most preferably, about 105%. The rough filter can be selected to remove at least a portion of components that are greater in diameter than about 60 μm, more preferably about 30 μm, even more preferably about 10 μm, or most preferably about 5 μm. Particularly well-suited filters for removal of rough components are filters with 5 μm pore size manufactured by Millipore.

The step of separation also includes directing the crude sample through a filter suitable for removing at least a portion of components that are smaller than the molecular species of interest, i.e., components that are smaller in diameter or molecular weight than the molecular species, for example salt components. Such filter is hereinafter referred to as a “fine filter”. Preferably, the fine filter removes components that have a molecular weight that is a fraction of the molecular weight of the molecular species. Preferably, said fraction is about 50%, more preferably about 75%, even more preferably about 90%, and most preferably, about 95%. Particularly well-suited filters for removal of fine components are manufactured by Millipore and provide a molecular weight cut-off of from about 10 to 60 kD. One skilled in the art will recognize that the separation steps can be conducted in any order.

The sample, wherein one or more molecular species have been separated from the rough component and, optionally, from the fine component, is hereinafter referred to as a “cleared sample”.

The acidity of the cleared sample can optionally be adjusted. Preferably, the acidity of the cleared sample is adjusted to be about two pH units below the acidity of a high-electrolyte buffer, defined below. For example, the acidity of the cleared sample is adjusted to between about 5 to about 7 pH units, preferably to between about 5.5 to about 6.5 pH units. Adjusting pH levels of the cleared sample permits concentration of the sample as will be described below.

In one embodiment of the present invention, at least one molecular species contained in the cleared sample can optionally be at least partially denatured.

As used herein, “denaturing” or “denaturation” means changing the conformation and/or the solubility of a molecular species to prepare it for detection. Denaturing and denaturation can be either partial or full. Partial denaturation means partial loss of native conformation of a molecular species and a change in solubility at least sufficient to prepare it for detection. Full denaturation means complete loss of native conformation of a molecular species and a change in solubility sufficient to prepare it for detection. For example, when the molecular species is a protein, denaturation can include transformation from a packed three-dimensional conformation to a linear conformation. Denaturation can also include solubilizing the macromolecule with the denaturing detergent. Denaturation can be accomplished by techniques well known to one skilled in the art, for example, addition of one or more denaturation agents, application of heat, disulfide bond reduction, or a combination thereof. Denaturation agents for proteins can include, for example, chaotropic agents e.g., urea, guanidine hydrochloride, and the like; detergents, e.g., sodium dodecyl sulfate, potassium laurel sulfate, and the like; disulfide cleavage agents, e.g. dithiothreitol, dithioerythritol, and the like; acids or bases, e.g., trichloroacetic acid, sodium 0.10 hydroxide, and the like; and other agents known to the art. Denaturation agents for polynucleic acids can include, for example, chelation agents, e.g., ethylenediamine tetraaceticacid and the like.

Denaturing can comprise adding to the cleared sample at least one detergent. Any ionic detergent can be used. Sodium dodecyl sulfate (SDS) is preferred. Denaturing can further comprise heating the sample in the presence of the detergent. Preferably, the cleared sample is heated for a period of time from about 1 minute to about 10 minutes at a temperature from about 60° C. to about 100° C. Heating for about 2 minutes at about 90° C. is preferred.

One skilled in the art will recognize that the steps of adjusting the acidity of the cleared sample and optionally denaturing at least one molecular species can be combined and achieved by admixing a sample buffer to the cleared sample followed by heating. The composition of the sample buffer can be determined by a skilled artisan based on the acidity of the crude sample and the expected concentration of the molecular species to be analyzed. In a preferred embodiment, the sample buffer used for acidic crude sample comprises about from 20 mM to about 100 mM of tris(Hydroxymethyl)aminomethane, from about 2% to about 10% of SDS, and from about 0% to about 20% glycerol. Preferably, the acidity of the sample buffer is adjusted to about two pH units below the pH of a high-electrolyte buffer described below. For example, if the high-electrolyte buffer contains Tris-HCl having a pKa of about 8.1-8.3, the final adjusted sample buffer composition can be: 62.5 mM Tris-HCl, 2% SDS, pH 6.4.

Optionally, partially denatured molecular species contained in the cleared sample are separated from insoluble contaminants by removing insoluble contaminants from the cleared sample. Any of the methods known in the art suitable for separation of soluble and non-soluble components can be used, for example centrifugation. Any of the commercially available centrifuges can be used, for example Bio-Rad® Mini Centrifuge Model SD™. For example, centrifugation can be performed at about 7000 g for about 30 seconds. Alternatively, filteration can be a substitute for centrifugation in the manual embodiment. Examples of filters that can be used to practice the present invention include Millipore filters (e.g., nylon filters and molecular weight) that remove at least a portion of components that are greater in diameter than about 60 μm, more preferably about 30 μm, and even more preferably about 5 μm, and provide molecular weight cut-offs from about 10 to about 100 kD.

In a preferred embodiment, an apparatus described in copending patent applications filed under the Ser. Nos. ______ attorney docket number 3555.1002-000 and 3555.1003-000 can be used for denaturing. The cleared sample is directed to a denaturation vessel, that can be any chamber or conduit where denaturation takes place, typically a small volume metal vessel, e.g., a stainless steel vessel between about 1 to about 100 mL. A denaturation vessel is typically coupled to a heating element, i.e., any device known to the art that can be used to heat the fluid mixture, for example, a resistive heating coil, a microwave heater, a combustion heater such as a gas flame, a heat pump, and the like. A denaturation vessel can also be coupled with a cooling element, for example, a heat pump, refrigeration unit, thermoelectric cooling element, radiator, water cooling coil, and the like. One skilled in the art will recognize that heating and cooling elements can be part of a single heat exchanger unit.

According to the method of the present invention, the cleared sample is introduced into a separation device, said device comprising: (i) a capillary, at least partially filled with a high-electrolyte buffer, said capillary having an inlet end and an outlet end; (ii) a means for applying voltage between the inlet end and the outlet end of said capillary; and (iii) a means for applying a pressure differential between the inlet end and the outlet end of said capillary.

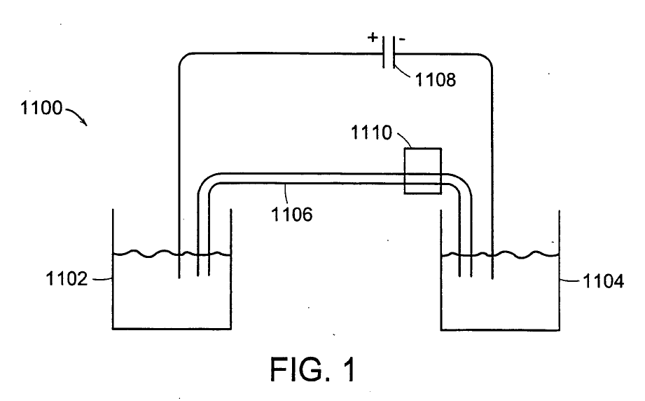

An example of a separation device is shown in FIG. 1. The separation is performed in a capillary tube having an internal diameter on the order of tens to hundreds of micrometers. In such small tubes the heat generated by the electric field is easily dissipated, so that high electrical fields can be used, leading to fast separations. FIG. 1 depicts a schematic of an electrophoresis apparatus 1100. An inlet vessel 1102 and an outlet vessel 1104 are connected by a capillary column 1106. The vessels and the capillary contain a buffer with an appropriate electrolyte. Upon loading a sample containing the analyte of interest at the inlet vessel, an electric field provided by a high voltage power supply 1108 causes the various molecules in the sample to separate, whereupon they can be detected by a detector 1110.

The capillary is packed with a porous separation media defined above. The high-electrolyte buffer composition is well known in the art and, for examples, can comprise 20% Dextran (MW 500,000), 0.2% SDS, 120 mM 2-amino-2-methyl-1-3-propanediol (AMPD)-cacodylic acid. Acidity of the high-electrolyte buffer is between about 7 and about 9 pH units, preferably between about 7.5 and 8.5 pH units, for example 8.8 pH units. In one preferred embodiment, a commercially available SDS Protein Separation Medium™ produced by Sigma-Aldrich can be used. In one embodiment, the ends of the capillary may be immersed into two separate reservoirs of the high-electrolyte buffer, buffer in one reservoir having been grounded. A high voltage power supply can provide potential difference between the inlet end and the outlet end of said capillary by having one electrode immersed into the non-grounded reservoir and another electrode grounded. A low-pressure (less than about 3 Bar) compressed gas, such as air or nitrogen, can be used a source of pressure differential between the inlet end and the outlet end of the capillary. The alternative is a vacuum or pressure source, such as a syringe pump.

In one preferred embodiment, the method of the present invention can be advantageously used in conjunction with a commercially available capillary electrophoresis system such as a Prince 2-LIFT™ model PrinCE 550™ produced by Prince Technologies®. In a preferred embodiment, commercially available linear polyacrylamide-coated capillaries having the internal diameter from about 40 μm to about 100 μm and length from about 50 cm to about 100 cm can be used (Bio-Rad).

The cleared sample can be introduced into the capillary using any of the known methods such as electokinetic injection or pressure injection. With electrokinetic injection, one end of the capillary and its electrode are removed from their buffer compartment and placed in a small cup containing the sample. A potential is then applied for a measured time, causing the sample to enter the capillary by a combination of ionic migration and electroosmotic flow. Pressure injection comprises creating a pressure differential between the sample and the capillary. Pressure injection is preferred.

Without being limited to any particular theory, it is believed that the time of injection Tᵢ and injection pressure Pᵢ satisfy the following equation (1):

Tᵢ×Pᵢ≦a×(T_{c}×P_{c}) (1)

where T_{c} is time required to fill the capillary with a buffer at pressure P_{c} and a is a number between 0 and 1 inclusive. Coefficient a is selected so that the injection time Tᵢ is sufficient to fill a pre-determined portion of the capillary.

In a preferred embodiment, the portion of the capillary to be filled with a sample is not greater than 10% of total volume resulting in a of about 0.1. For example, the cleared sample is injected into the capillary under about 500-2500 mBar of pressure. Pressure differential is maintained for about 0.5 minute.

In another preferred embodiment, an apparatus described in a co-pending application filed under the Ser. No. ______ attorney docket number 3353.1003-000 can be used. The capillary electrophoresis apparatus can be automatically controlled to conduct the necessary steps. The apparatus for capillary electrophoresis includes a plurality of inlet chamber connected to capillary electrophoresis columns. One end of each column is fixed at the interior of the inlet chamber. Preferably, the column has a length of at least about 20 centimeters, more preferably at least about 30 centimeters, and most preferably, at least about 50 centimeters. Also included is a liquid source adapted for automatic control. The liquid source supplies a liquid sample through an input valve into the inlet chamber so that the sample is in fluid communication with the end of the column. The inlet chamber is supplied with the liquid sample by a pump through an inlet valve from a liquid sample source. Optional precipitate filter can be employed to separate insoluble contaminants from the liquid sample by employing a pump to apply the liquid sample to the precipitate filter with a pressure differential across the filter. Particularly well-suited precipitate filters are filters with 0.45 μm pore size manufactured by Millipore.

Each chamber can be supplied independently by a buffer from a reservoir through valves and can be independently drained. A pump can draw filtered air through air inlet valve from an air source, and independently direct the air to chambers.

In a preferred embodiment, the step of introducing the cleared sample into a separation device includes introducing a low-electrolyte, low-conductivity plug into the capillary. The low-electrolyte plug is introduced into the capillary before introduction of the cleared sample. The introduction of the low-electrolyte plug into the capillary can be accomplished in the same manner as the introduction of the sample as described above.

The volume of the low-electrolyte plug is preferably between about 10 to about 20% of the capillary volume and most preferably would be between about 1 to about 2 times the volume of the injected sample. Equation (1) can be used to calculate time and pressure required for the low-electrolyte plug injection wherein a is between about 0.1 and about 0.2. For example, pressure injection into the capillary can be used wherein the low-electrolyte plug is injected under about 500 mBar of pressure for about 1 minute.

The low-electrolyte plug is located between the high-electrolyte buffer and the cleared sample at the inlet end of the capillary. A boundary exists between the low-electrolyte plug and the high-electrolyte buffer. Preferably, the low-electrolyte plug has either ionic strength or acidity or both that is substantially different from that of the high-electrolyte buffer. As used herein, the two solutions have substantially different ionic strength if the total ion strengths (total ion concentration) of the two solutions differ by a factor of at least about 5, preferably 10. As used herein, two solutions have substantially different acidity if the difference between their respective acidities is at least one half, preferably one pH unit. For example, the high-electrolyte buffer has an acidity of about 8.3 pH units and the ionic strength of about 120 mM and the low-electrolyte plug has an acidity of about 7.0 pH units and the ionic strength of 12 mM. Preferably, the low-electrolyte buffer consists essentially of water and has ionic strength of about zero.

In a particularly preferred embodiment, the cleared sample can further comprise molecular weight standards. Any of the commercially available molecular weight standards can be used, for example albumin from bovine serum, Sigma-Aldrich catalog number P-0914. The molecular weight standards can be admixed to the cleared sample prior to the injection into the capillary or can be injected into the capillary after the injection of the low-electrolyte plug.

According to the method of the present invention, the cleared sample that has been introduced into the separation device can be pre-focused. As used herein, “pre-focusing” comprises stacking the molecular species of interest in the cleared sample into a band, followed by application of negative pressure to push the stacked sample to the original starting position.

As used herein, “sample stacking” refers to a sample concentration technique wherein a long plug of low-electrolyte buffer or water is introduced into the capillary filled with a high-electrolyte buffer prior to introduction of a sample. After the sample is introduced, a voltage is applied between the inlet end and the outlet end of the capillary that causes electrophoretic migration. Without being limited to any particular theory, it is believed that the low-electrolyte buffer possesses a higher resistivity, and consequently the electric field is stronger in the low-electrolyte buffer than in the high-electrolyte buffer. As a result, the molecular species and other charged molecules migrate from the sample buffer into the low-electrolyte buffer and then rapidly continue to migrate toward the boundary that exists between the low-electrolyte and the high-electrolyte buffers. At the boundary between the low-electrolyte and the high-electrolyte buffers, they experience a weaker electrical field and consequently slow down. This process results in formation of narrow and concentrated bands, or stacks, of the molecular species to be analyzed. Sample stacking is described in Burgi D. S. and Chien R.-L. “Optimization in Sample Stacking for High-Performance Capillary Electrophoresis” Anal. Chem. (1991) 63:2042-2047, Chien R.-L and Burgi D. S. “Sample Stacking of an Extremely Large Injection Volume in High-Performance Capillary Electrophoresis” Anal. Chem. (1992) 64:1046-1050, and Shihabi Z. K. “Stacking in capillary zone electrophoresis” J. Chromatogr. (2000) A 902:107-117, the entire teachings of which are herein incorporated by reference. Stacking permits concentration of low-abundance molecular species. Furthermore, stacking forces the molecular species into a narrow band within the capillary. Formation of the concentrated and narrow bands of the molecular species allows quantitative and qualitative monitoring of reaction of interest both by enhancing detection threshold and improving resolution.

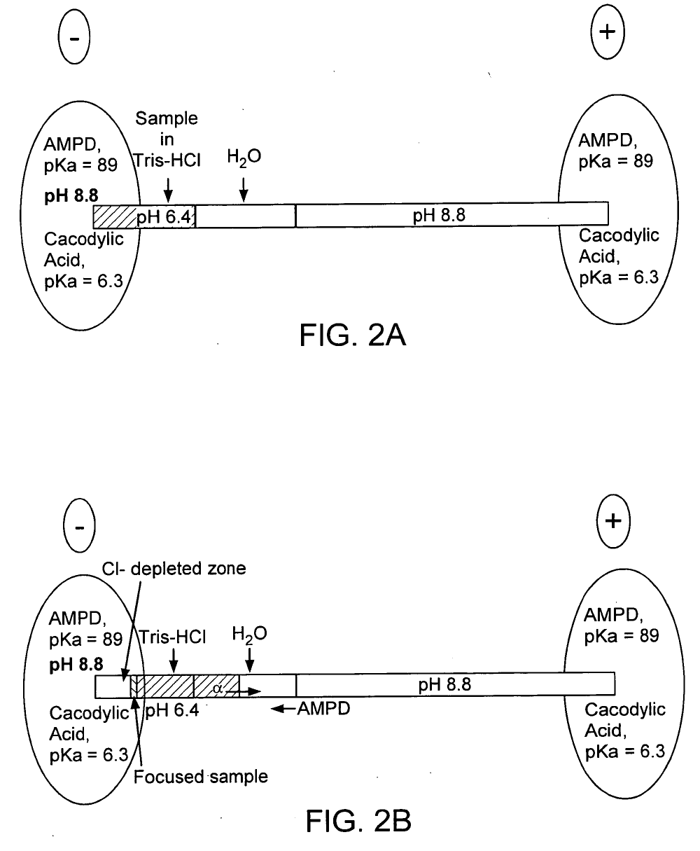

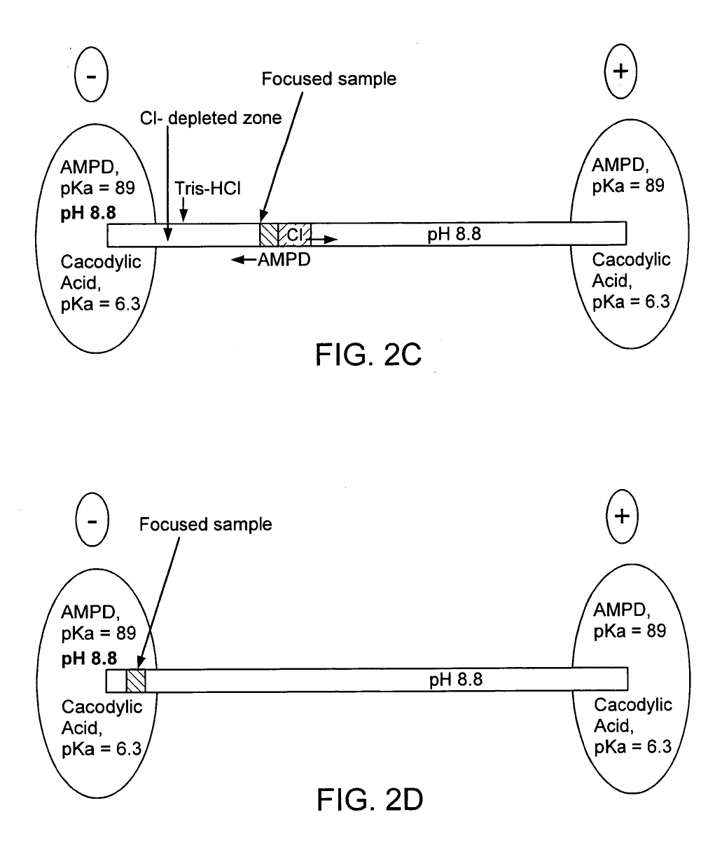

According to the method of the present invention, after the cleared sample has been stacked, a negative pressure is applied between the inlet end and the outlet end of the capillary to push the concentrated band of the molecular species back to its original starting position and to remove the excess fluid, including low-electrolyte buffer from the capillary. Thus, pre-focusing comprises applying voltage between the inlet end and the outlet end of the capillary. The voltage is preferably applied for a period of time sufficient to cause electrophoretic migration of at least one molecular species from the cleared sample up to the boundary between the low-electrolyte and the high-electrolyte buffers. Pre-focusing further comprises applying a pressure differential between the inlet end and the outlet end of the capillary. The pressure is applied for a period of time sufficient to remove the excess sample buffer from the capillary and to cause the pre-focused molecular species to be pushed toward the inlet end of the capillary. Preferably, the pressure is applied for a period of time sufficient to remove from the capillary the excess sample buffer and a portion of a low-electrolyte plug adjacent to the sample buffer that does not include the concentrated band of molecular species of interest. Pressure differential between the inlet end and the outlet end of the capillary that causes a content of the capillary to move toward the inlet end of the capillary is referred to as “negative pressure”. Pushing of the molecular species toward the inlet end of the capillary is referred to as “backward displacement”. The schematic depiction of pre-focusing is shown in FIG. 2A through 2B.

Thus, the method of preparing a crude sample for detecting at least one molecular species of interest comprises:

(a) acquiring a crude sample containing at least one molecular species, at least one crude component, larger than the molecular species, and at least one fine component smaller than the molecular species;

(b) separating the rough component and, optionally, the fine component from the molecular species, thereby producing a cleared sample, optionally adjusting the acidity of the cleared sample comprising the at least one molecular species of interest;

(c) optionally, at least partially denaturing at least one molecular species in the cleared sample;

(d) optionally, removing insoluble contaminants from the cleared sample;

(e) introducing the cleared sample into a separation device; and

(f) pre-focusing at least one molecular species in the crude sample.

In a preferred embodiment, the above-described steps are performed one or more times. The number of times the above-described steps are performed is preferably sufficient to bring the concentration of at least one molecular species in the sample up to either a level of detection or a level of quantitation. As used herein, the “level of detection” (LOD) is defined as at least three times the level of a detector noise. As used herein, the “level of quantitation” (LOQ) is defined as ten times the level of a detector noise. These definitions comply with international standards such as ASTM 685. A skilled artisan will readily determine the number of times sufficient to bring the concentration of at least one molecular species in the sample up to the level of detection based on the expected concentration of a species to be detected and a method of detection. Repeated application of pre-focusing makes analysis of very dilute large volumes of crude samples possible. Preferably, pre-focusing is repeated a number of times sufficient to cause the migration of the first concentrated band of molecular species up to a boundary that exists between the low-electrolyte plug and the high-electrolute running buffer. For example, pre-focusing can be repeated up to about 4 to about 5 times, preferably about 4 or 5 times.

Negative pressure can be applied either after stacking of each aliquot of cleared sample or once after the desired number of aliquots has been stacked. Without being limited to any particular theory, it is believed that the repeated application of voltage without repeated application of negative pressure can cause formation of excessive volume of the low-electrolyte plug and diffusion high-electrolyte buffer into the low-electrolyte plug. Thus, it is preferable that negative pressure be applied after stacking of each aliquot of cleared sample.

Without being limited to any particular theory, it is believed that the time period T_{pf} sufficient to cause electrophoretic migration of at least one molecular species in the cleared sample into the low-electrolyte plug, voltage V applied between the inlet end and the outlet end of the capillary and length L of the capillary satisfy the following equation (2):

T_{pf}×V/L≈constant (2)

As a non-limiting example, a cleared sample can be pre-focused by applying a voltage of about −30 kV between the inlet end and the outlet end of a capillary having a length of about 80 cm for a period of about 3 minutes. Similar results can be achieved by applying a voltage of about −15 kV between the inlet end and the outlet end of a capillary having a length of about 40 cm for a period of about 3 minutes.

Without being limited to any particular theory it is believed that the time T of delivering negative pressure and the value P of the negative pressure satisfy the following equation (3):

T×P≦T_{w}×P_{w}+Tₛ×Pₛ (3)

where T_{w} is the time of delivering the low-electrolyte plug, P_{w} is a positive pressure used to deliver the low-electrolyte plug, Tₛ is the time of delivering a sample and Pₛ is a positive pressure used to deliver the sample.

Preferably, the total volume of the backward displacement of the sample band is equal or less than the total volume of the low-electrolyte plug and the sample plug. Most preferably, the value of the product, T×P is from about 80 to about 90% of the value of the sum T_{w}×P_{w}+Tₛ×Pₛ. It is further believed that such parameters as the diameter and length of a capillary, composition of the sieving medium, voltage and time of stacking can be optimize alone or in combination to achieve the best results.

As a non-limiting example, when a commercially available capillary electrophoresis system such as a Prince 2-LIFT™ model PrinCE 550™ is used, voltage in the range of from about −10 kV to about −60 kV can be applied for a period of time from about 0.5 minute to about 10 minutes. Preferably, voltage from about −25 kV to about −35 kV is applied for a period of time from about 0.5 minute to about 10 minutes. More preferably, voltage of about −30 kV is applied for about 3 minutes. The negative pressure of between about −0.75 psi and about −40 psi is applied for a period of time from about 0.5 minute to about 10 minutes. Preferably, the negative pressure of between about −2 psi and about −40 psi is applied for a period of time from about 1 minute to about 5 minutes. More preferably, the negative pressure of about −3 psi is applied for about 3 minutes.

In another embodiment, the present invention is a method of detecting at least one molecular species in a crude sample. After the crude sample has been prepared as described above, the sample is ready for electrophoretic separation and detection.

Any of the electrophoretic techniques known in the art can be used in conjunction with the method of the present invention. In one embodiment, the electrophoretic separation of the molecular species in the cleared sample can be performed by the separation device as described above, adapted to employ at least one of the methods selected from the group consisting of capillary zone electrophoresis (CZE), capillary gel electrophoresis (CGE), capillary isoelectric focusing (CIEF), and capillary isotachophoresis (CITP). In capillary zone electrophoresis method, the buffer composition is constant throughout the region of the separation, while in capillary isoelectric focusing method a gradient of acidity and/or ionic strength exists across the zone of separation. Capillary gel electrophoresis is generally performed in a porous gel polymer matrix, the pores of which contain a buffer mixture in which the separation is carried out. In capillary isotachophoresis all analyte bands ultimately migrate at the same velocity. Capillary gel electrophoresis is preferred. Electrophoretic separation of the molecular species comprises applying voltage between the inlet end and the outlet end of the capillary for a period of time sufficient to separate the molecular species in the sample. As a non-limiting example, voltage in the range from about −10 kV to about −60 kV can be applied for a period of time from about 5 minute to about 90 minutes. Preferably, voltage from about −25 kV to about −35 kV is applied for a period of time form about 5 minutes to about 30 minutes. More preferably, voltage of about −30 kV is applied for about 20 minutes.

According to the method of the present invention, the separated molecular species can be detected by any of the methods of detection known in the art, including techniques other than electrophoresis. Detecting can be performed either before or after elution of the separated molecular species from the capillary.

The methods of detection suitable for use with the present invention include both direct and indirect absorbance methods, fluorescent methods and electrochemical detection, such as conductivity measurement and amperometry. In direct absorbance methods, radiation of specific wavelength is absorbed by a molecular species to be detected and the corresponding absorbance is measured. Indirect absorbance detection can be used for detection of species that are difficult to detect because of low molar absorptivities without derivatizaton. An ionic chromophore is placed in the electrophoresis buffer. Electrochemical methods are based on detecting the presence of the desired molecular species by measuring the changes in the conductivity of the electrolyte buffer. Methods of detection suitable for practicing the present invention are described, for example, in Kaul, R. and Mattiason, B., Bioseparation (1992) 3:1-26 and in Ance, L., et al. Analytica Chimica Acta (1999) 390:34-44, the entire teachings of which are incorporated herein by reference.

The step of detecting includes providing a detector, which, in one embodiment, can be a UV absorbance detection device. In another embodiment, detecting the separated molecular species comprises providing a light source for exciting fluorescence in the separated protein species and a detector for detecting said fluorescence. The light source can be a laser. In one embodiment, a commercially available UV absorbance detector can be used, such as, for example, a Bischoff® model Lambda 1010™ UV detector. In one embodiment, the detector is a mass spectrometer, wherein the outlet end of the capillary of the separation device is coupled directly to the ionization source of a mass spectrometer. The separated molecular species ionization technique used for this purpose can be an electrospray or fast atom bombardment. In a preferred embodiment, an apparatus described in copending patent application filed under the Ser. No. ______ attorney docket number 3555.1003-000 can be used for detection. The apparatus disclosed in the above-referenced application can, optionally, include a detector, that can implement any detection method known in the art of detection of molecular analytes. For example, the optional detector can implement detection by absorbance/transmission of radiation, e.g., ultraviolet/visible light; fluorescence detection; refractive index detection; electrochemical detection; mass spectrometric detection; detection of electron or nuclear magnetic resonance; flame ionization detection; binding, e.g., in an enzyme or antibody assay; detection of a spectroscopic or radioactive label; and the like. When the optional detector is an optical detector, it can be configured to detect molecular analytes that are inside the capillaries. Alternatively, fractions of the eluents can be collected at the outlet ends of the capillaries and the fractions can be further analyzed separately.

In another preferred embodiment, the capillary electrophoresis apparatus described in a co-pending application filed under the Ser. No. ______ attorney docket number 3353.1003-000 can be used to conduct the necessary steps under automatic control. An automated controller can be a processor, e.g., an embedded processor, a desktop computer, and the like, that can be programmed to control a system adapted for automatic control, e.g., the hydraulic system.

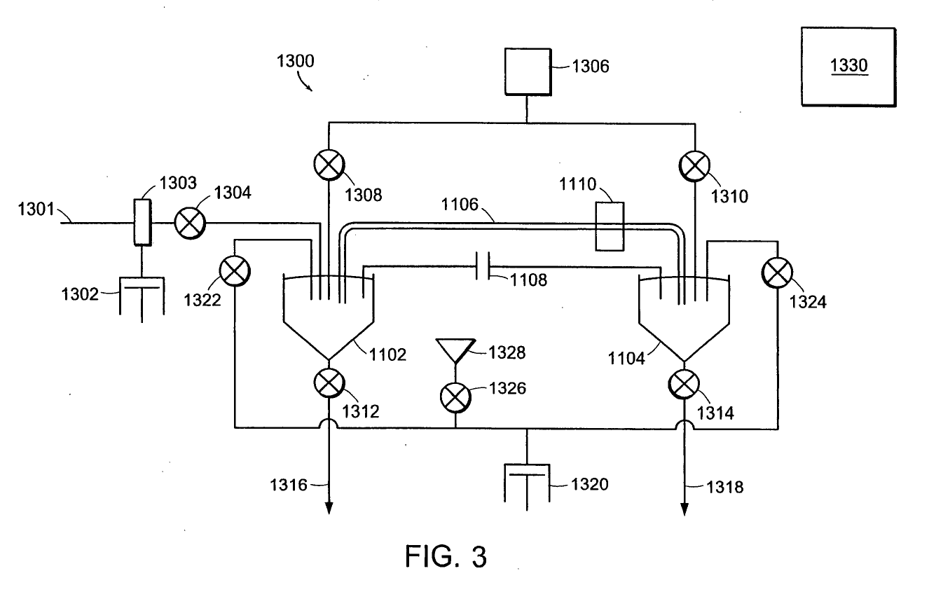

FIG. 3 depicts a stationary capillary electrophoresis circuit 1300 that can be controlled to conduct the steps the method of the present invention. The inlet chamber 1102 is supplied with the liquid sample by pump 1302 through inlet valve 1304 from liquid sample source 1301. Optional precipitate filter 1303 can be employed to separate insoluble precipitates from the liquid sample by employing pump 1302 to apply the liquid sample to filter 1303 with a pressure differential across the filter.

Each chamber can be supplied independently by a buffer reservoir 1306 through valves 1308 and 1310. Each chamber can be independently drained via valves 1312 and 1304 to waste sites 1316 and 1318. Pump 1320 can draw filtered air through air inlet valve 1326 and air source 1328, and independently direct the air to chambers 1102 and 1104 through valves 1322 and 1324. The valves, pumps, optional electrophoresis power supply 1108, and optional detector 1110 are adapted to be controlled by an optional controller 1330.

The capillary electrophoresis column 1106 is coupled with the interior of each chamber so that liquid in each chamber can be placed in fluid communication with the respective end of column 1106. Preferably, the column has a length of at least about 20 centimeters, more preferably at least about 30 centimeters, and most preferably, at least about 50 centimeters.

The optional detector 1110 can be any detection method known to the art for detection of molecular analytes, for example, absorbance/transmission of radiation, e.g., ultraviolet/visible light; fluorescence detection; refractive index detection; electrochemical detection; mass spectrometric detection; detection of electron or nuclear magnetic resonance; flame ionization detection; binding, e.g., in an enzyme or antibody assay; detection of a spectroscopic or radioactive label; and the like. When optional detector 1110 is an optical detector, it can be configured to detect molecular analytes that are inside column 1106. Or, fractions can be collected from the molecular analytes exiting column 1106, e.g., at outlet chamber 1104, and the fractions can be analyzed separately from the column.

Additionally, each chamber can be barometrically sealed, i.e., they can be pressurized or depressurized. For example, valves 1304, 1308, 1312, 1324, and 1326 can be closed, valve 1322 can be opened, and pump 1320 can pressurize inlet chamber 1102. If the pressure in chamber 1102 is greater than the pressure in outlet chamber 1104, a high to low pressure differential results across the length of capillary electrophoresis column 1106. Alternatively, pump 1320 can reduce the pressure in chamber 1102 to less than the pressure in chamber 1104, resulting in a low to high pressure differential, which can direct liquid from chamber 1102 through column 1106 to chamber 1104. Or, the valves can be configured so that pump 1320 can pressurize or depressurize chamber 1104. Optionally, separate independent pumps can be coupled with each chamber and the pumps can operate cooperatively, one pulling and the other pushing, to create a pressure differential across column 1106. Creation of a pressure differential between chamber 1102 to chamber 1104 through column 1106 can be employed to fill, purge, or clean the column, or to move fluid through the column, e.g., perform step 1214.

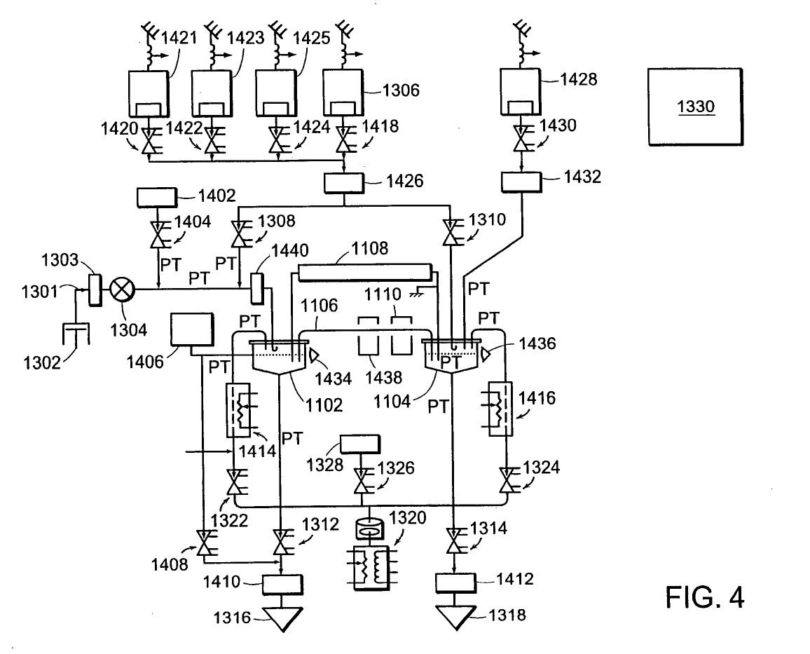

FIG. 4 depicts a more detailed schematic of the capillary electrophoresis circuit. The line between inlet valve 1304 and inlet chamber 1102 can be supplied with compressed air by filtered air supply 1402 through valve 1404. An additional optional air source 1406 and valve 1408 is provided that can be employed to purge inlet chamber 1102 and/or the waste line between valve 1312 and waste 1316. The waste from both chambers is provided with flow sensors 1410 and 1412, respectively. Pressure transducers 1414 and 1416 are provided to sense the pressure in the apparatus. Along with reservoir 1306 are provided a valve 1418 and additional valved reservoirs 1420/1421, 1422/1423, and 1424/1425. These reservoirs can supply water, buffer, cleaning solution, solvents, electrolytes, and the like, the flow of which can be sensed at flow sensor 1426. Additional buffer can be supplied to the outlet chamber 1104 by reservoir 1428 through flow sensor 1430 and valve 1432.

Additionally, the level of fluid in chambers 1102 and 1104 can be sensed independently by level sensors 1434 and 1436, respectively. Heat generated in column 1106 by the electrophoresis current can be removed by a heat exchanger 1438, which can be, for example, a cooling element, a thermoelectric element, and the like. Also, optional degas unit 1440 can be employed to remove at least a portion of dissolved gases.

### EXEMPLIFICATION

The present invention will now be illustrated by the following Examples, which are not intended to be limiting in any way.

### Example 1

### SDS-Capillary Gel Electrophoresis is Used to Detect Protein in Sequentially Timed Crude Samples

The capillary equipment used for this assay was a Prince® model PrinCE 550™ capillary electrophoresis instrument equipped with a Bio-Rad® LPA-coated capillary (50 μm i.d.×80 cm with detection window at 31 cm from injection end). Detection was performed with a Bischoff® model Lambda 1010™ UV detector at 214 nm. The buffer electrolyte was either 45% or 50% diluted Sigma-Aldrich® SDS Protein Separation Medium™ (10% Dextran (MW500,000), 0.1% SDS, 60 mM 2-amino-2-methyl-1-3-propanediol (AMPD)-cacodylic acid, pH 8.8). All results were acquired and reduced using DAX™ software supplied by Prince Technology®. The slab gel electrophoresis equipment, Mini-PROTEAN 3™, was obtained from Bio-Rad®. The gels were pre-cast and contained the percentage of polyacrylamide as indicated in the report. Dyes, Coomassie Blue and silver stain, were acquired from Bio-Rad®. Capillary electrophoresis was performed at 22° C. using a negative polarity of −30 kV.

Samples consisted of a reference aliquot (Standard) and sequential timed aliquots taken from a fermentor batch. Sets of protein molecular weight standard were obtained from Sigma-Aldrich® and Bio-Rad®.

Molecular weight standards were assayed on the CGE system and on slab gel to calibrate molecular weight as a function of retention time in the CGE apparatus or migration distance in slab gel. The Standard and timed aliquots were consequently assayed on CGE and simultaneously assayed on slab gel.

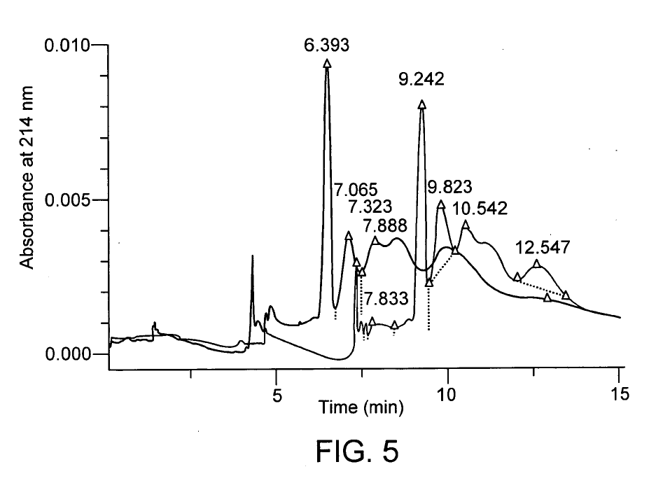

Microplasminogen aliquots (200 μL) were mixed with 200 μL of native sample buffer (62.5 mM Tris-HCl, pH 6.8, 40% glycerol, 0.01% Bromophenol blue (Bio-Rad®)) and 100 μL of 5× sample buffer A (312.5 mM Tris-HCl, 10% SDS, pH 6.8) to the final concentration of 87.5 mM Tris-HCl, 2% SDS, and 16% glycerol. The samples were injected (approximately 0.30 μL of sample for 30 second at 500 mbar) and ran at −30 kV. FIG. 5 shows a comparison of electropherograms of a 1:4 mixture of aliquot No. 1 and Sigma® molecular weight markers prepared with (bold trace) and without (thin trace) sample stacking steps. Microplasminogen has a molecular weight of 28 kD and would be benefited by the sample stacking step. Sample stacking steps were included in all subsequent purification runs.

The timed 50 μL aliquots containing recombinant plasminogen were mixed with 5× sample buffer to obtain the final concentration of 87.5 mM Tris-HCl, 2% SDS, and 16% glycerol. Upon neutralization, the solution turned turbid and so were centrifuged for 30 second at 6000 rpm (7000 g) using Bio-Rad® “Mini Centrifuge Model SD”. The precipitate was separated from the solution and re-dissolved in a sample buffer corresponding to one-third of the original volume of the solution resulting in a three-fold concentration of the sample. The sample stacking program is described below and was optimized to give sharper peaks.

Quantifying the plasminogen content of the aliquots was carried out using a 75 μm i.d.×80 cm capillary. The composition of the sample buffer used for quantifying was 312.5 mM Tris-HCl, 10% SDS, pH 8.0. Microplasminogen aliquots No.1-No. 6 were individually mixed 4:1 with the sample buffer and have maintained 80% of its original concentration.

The conditions for operating the CE system are described below:

| | | |
|---|---|---|
| Prime line: | 2000 | mBar, 1 min |
| Water: | 500 | mBar, 1 min |
| Injection: | 500 | mBar, 0.5 min |
| Focus: | −30 | kV, 3 min |
| Back Flush: | −180 | mBar, 3 min |
| Run: | −30 | kV, 14 min |

Peak areas of the microplasminogen were plotted against their known concentrations to obtain the standard curve.

### Example 2

### SDS-Capillary Electrophoresis Permits Rapid and Sensitive Detection of Crude Recombinant Protein

The capillary equipment used for this assay was a Prince 2-LIFT™ model PrinCE 550™ capillary electrophoresis instrument equipped with an autosampler. Bio-Rad® LPA-coated capillaries were used (length 80 cm; internal diameter (i.d.) 75 μm or 50 μm, detection window at 30.5 cm from injection end). Detection was performed with a Bischoff® Lambda 1010™ UV detector at 214 nm (SDS-CE System). The buffer electrolyte was 45% diluted SDS Protein Separation Medium obtained from Sigma-Aldrich®. All results were acquired and reduced using DAX™ software supplied by Prince Technology®. The slab gel electrophoresis equipment, Mini-PROTEAN 3™, was obtained from Bio-Rad®. The gels were pre-cast and contained the percentage of polyacrylamide as indicated. Coomassie Blue and silver stain, were acquired from Bio-Rad®. All capillary electrophoresis was performed at 22° C. using a negative polarity of −30 kV.

The samples consisted of sequential timed aliquots of a purified murine recombinant protein Placenta Growth Factor (rMuPlGF) taken from fermentor batch runs. Molecular weight markers were obtained from Sigma-Aldrich® and Bio-Rad®. Samples were mixed 1:1 with 2× sample buffer obtained from Sigma® and heated in boiling water for 5 minutes before use. Sample buffer composition used was:

125 mM Tris-HCl, 4% SDS, 20% glycerol, 10% 2-mercaptoethanol, 0.004% bromophenol blue, pH 6.8.

Sieving medium (10% Dextran (MW500,000), 0.1% SDS, 60 mM 2-amino-2-methyl-1-3-propanediol (AMPD)-cacodylic acid, pH 8.8) was pressurized and delivered through the capillary at 2000 mbar. Samples were injected 0.1 min at 500 mbar pressure unless noted otherwise. Water plug for sample concentration was delivered at 1000 mbar and removed using reversed pressure at −165 mbar. At the end of each run, the sieving medium was removed and the capillary was re-supplied with fresh medium for 5 minutes.

Molecular weight standards were assayed on the SDS-CE system and on slab gel to calibrate molecular weight as a function of retention time in the SDS-CE apparatus or migration distance in slab gel. Aliquots of the samples were consequently assayed on CE and simultaneously assayed on slab gel. The molecular weight markers information was used to identify the molecular weights of proteins in the samples based on retention time or migration distance.

SDS-CE sample stacking method was first tested on the sample of molecular weight markers in order to improve the detection of rMuPlGF in fermentation products. For these tests, a 50 μm i.d. of Bio-Rad® LPA-coated capillary was used.

A one minute injection of water plug was carried out prior to injection of samples. The volume of the water plug was approximately 15% of the column volume. Following the injection of samples, a sample focusing step was carried out by applying −30 kV of voltage for about 2 to 3 minutes. Next, a low negative pressure (about one third of injection pressure) was applied from the outlet end of a capillary for as long as about three times the sample injection time. The negative pressure pushed back the sieving medium and removed the water plug.

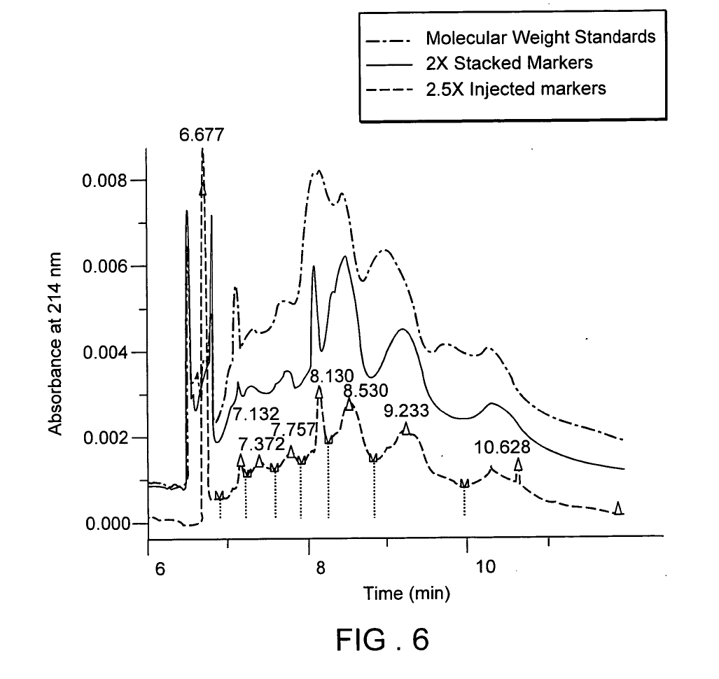

FIG. 6 shows the effects of sample stacking using the method described herein. Due to the low resolution of the protein bands, original molecular marker profile (bottom curve) was overloaded and the peaks broadened significantly if the injected volume was increased 2.5 fold (top curve). When the sample stacking method was applied, an improved resolution was achieved for similar injected volumes (middle curve). When the marker solution was diluted 10 fold and a one minute stacking injection was carried out, an electrophoresis profile essentially identical to the unstacked and undiluted markers was obtained, indicating a nearly ten fold increase in resolution (data not shown).

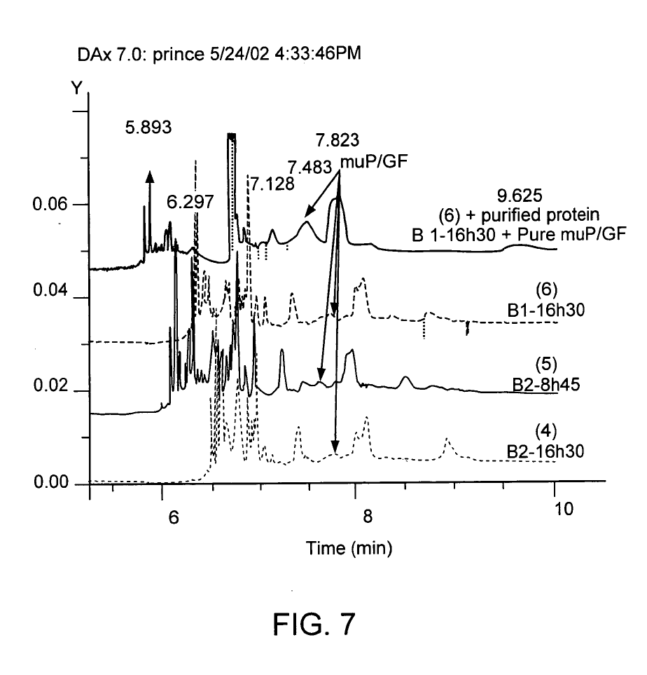

Fermentation products were then analyzed on the PrinCE 550™ system. As indicated in FIG. 7, the rMuPlGF protein was clearly identified in the crude samples. Electropherograms of fermentation aliquots No. 4-6 were overlayed for easy comparison. A run of a 1:1 mixture of sample No. 6 and the purified rMuPlGF was overlayed to identify the location of the rMuPlGF peak. For quantifying, rMuPlGF protein in the crude products was calibrated against a known concentration of bovine serum albumin. The ranges of concentrations obtained were at 10-30 μg/mL were consistent with the known immunoassay results.

Example 2 demonstrates the feasibility of using a SDS-CE system in conjunction with pre-focusing technique, to rapidly assay a series of dilute timed aliquots of fermentation reaction mixture.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of preparing a crude sample for detecting at least one molecular species of interest, comprising:
(a) acquiring a crude sample containing at least one molecular species of interest, one or more rough components that are larger than the molecular species of interest, and one or more fine components that are smaller than the molecular species of interest;
(b) separating from the molecular species of interest at least a portion of the rough component and, optionally, at least a portion of the fine component of the crude sample, thereby producing a cleared sample comprising the at least one molecular species of interest;
(c) optionally, at least partially denaturing the at least one molecular species of interest contained in a cleared sample; and
(d) pre-focusing the at least one molecular species of interest in the cleared sample, wherein steps (a) through (d) are performed for a number of times sufficient to bring the concentration of at least one molecular species of interest in the sample up to the level of detection.

2. The method of claim 1 wherein step (c) further comprises removing insoluble contaminants.

3. A method of detecting at least one molecular species of interest, comprising:
(a) acquiring a crude sample containing at least one molecular species of interest, one or more rough components that are larger than the molecular species of interest, and one or more fine components that are smaller than the molecular species of interest;
(b) separating from the molecular species of interest at least a portion of a rough component and, optionally, at least a portion of the fine component of the crude sample, thereby producing a cleared sample comprising the at least one molecular species of interest;
(c) optionally, at least partially denaturing the at least one molecular species of interest contained in a cleared sample;
(d) pre-focusing the at least one molecular species of interest in the cleared sample, wherein steps (a) through (d) are performed for a number of times sufficient to bring the concentration of the at least one molecular species of interest in the sample up to the level of detection;
(e) electrophoretically separating the molecular species of interest in the cleared sample; and
(f) detecting the separated molecular species of interest.

4. The method of claim 3 wherein step (c) further comprises removing insoluble contaminants.

5. The method of claim 3 wherein the molecular species of interest is selected from the group consisting of antibodies, proteins, peptides, peptidomemetics, peptide-nucleic acids, oligonucleotides, aptamers, lipids, polysaccharides, liposaccharides, lipoproteins, glycoproteins and small molecules.

6. A method of detecting at least one molecular species of interest, comprising:
(a) acquiring a crude sample containing at least one molecular species of interest, one or more rough components that are larger than the molecular species of interest, and one or more fine components that are smaller than the molecular species of interest;
(b) separating from the molecular species of interest at least a portion of a rough component and, optionally, at least a portion of the fine component of the crude sample, thereby producing a cleared sample comprising the at least one molecular species of interest;
(c) introducing the cleared sample into a separation device, said device comprising:
(i) a capillary, at least partially filled with a high-electrolyte buffer, said capillary having an inlet end and an outlet end;
(ii) a means for applying voltage between the inlet end and the outlet end of said capillary; and
(iii) a means for applying pressure differential between the inlet end and the outlet end of said capillary;
(d) pre-focusing the at least one molecular species of interest in the cleared sample, wherein steps (a) through (d) are performed for a number of times sufficient to bring the concentration of the at least one molecular species of interest in the sample up to the level of detection;
(e) electrophoretically separating the molecular species of interest in the cleared sample; and
(f) detecting the separated molecular species of interest.

7. The method of claim 6 wherein step (b) comprises filtering or centrifuging the sample.

8. The method of claim 6 wherein step (b) further comprises adjusting acidity of the sample to between about 5 and about 7 pH units.

9. The method of claim 8 wherein acidity of the sample is adjusted to about 5.5 to about 6.5 pH units.

10. The method of claim 6 wherein step (b) optionally includes at least partially denaturing the at least one molecular species of interest in the cleared sample.

11. The method of claim 10 wherein denaturing comprises adding to the cleared sample at least one denaturing agent selected from the group consisting of urea, guanidine hydrochloride, sodium dodecyl sulfate potassium lauryl sulfate, dithiothreitol, dithioerythritol, trichloroacetic acid, sodium hydroxide, ethylenediamine tetraaceticacid and combinations thereof.

12. The method of claim 10 wherein denaturing comprises adding to the cleared sample at least one detergent and heating the sample in the presence of said detergent.

13. The method of claim 12 wherein the sample is heated for a period of time from about 1 minutes to about 10 minutes at a temperature from about 60° C. to about 100° C.

14. The method of claim 13 wherein the sample is heated for about 2 minutes at about 90° C.

15. The method of claim 12 wherein at least one detergent is sodium dodecyl sulfate.

16. The method of claim 10 wherein step of separating from the molecular species of interest at least a portion of a rough component and, optionally, at least a portion of the fine component of the crude sample further comprises filtering or centrifuging the cleared sample.

17. The method of claim 6 wherein step (c) includes introducing a low-electrolyte plug into said separation device, said low-electrolyte plug disposed between the high-electrolyte buffer and the sample at the inlet end of the capillary, thereby forming a boundary between the low-electrolyte plug and the high-electrolyte buffer.

18. The method of claim 17 wherein said low-electrolyte plug has an acidity that is substantially different from that of the high-electrolyte buffer.

19. The method of claim 17 wherein the acidity of the high-electrolyte buffer is between about 7 and about 9 pH units.

20. The method of claim 17 wherein the acidity of the high-electrolyte buffer is about 7.5 to about 8.5 pH units.

21. The method of claim 17 wherein the low-electrolyte buffer consists essentially of water.

22. The method of claim 17 wherein step (d) comprises:
(i) applying voltage between the inlet end and the outlet end of the capillary for a period of time sufficient to cause electrophoretic migration of at least one molecular species in the cleared sample up to the boundary between the low-electrolyte plug and the high-electrolyte buffer, thereby pre-focusing and, optionally, desalting the cleared sample; and
(ii) applying a negative pressure differential between the inlet end and the outlet end of the capillary for a period of time sufficient to cause the pre-focused molecular species of interest to be pushed substantially toward the inlet end.

23. The method of claim 6 wherein step (e) is performed using at least one of the methods selected from the group consisting of capillary zone electrophoresis (CZE), capillary gel electrophoresis (CGE), capillary isoelectric focusing (CIEF) and capillary isotachophoresis (CITP).

24. The method of claim 23 wherein separating the molecular species of interest comprises applying voltage between the inlet end and the outlet end of the capillary for a period of time sufficient to separate the molecular species of interest in the sample.

25. A method of detecting at least one molecular species of interest in a sample, comprising:
(a) acquiring a crude sample containing at least one molecular species of interest, at least one rough component, larger than the molecular species of interest, and at least one fine component smaller than the molecular species of interest;
(b) separating the rough component and, optionally, the fine component from the molecular species of interest, thereby producing a cleared sample, thereby producing a cleared sample comprising the at least one molecular species of interest;
(c) optionally, adjusting the acidity of the cleared sample;
(d) optionally, at least partially denaturing the at least one molecular species of interest in the cleared sample;
(e) optionally, removing insoluble contaminants from the cleared sample;
(f) introducing the cleared sample into a separation device, said device comprising:
(i) a capillary, at least partially filled with a high-electrolyte buffer, said capillary having an inlet end and an outlet end;
(ii) a means for applying voltage between the inlet end and the outlet end of said capillary; and
(iii) a means for applying pressure differential between the inlet end and the outlet end of said capillary;
(g) pre-focusing the at least one molecular species of interest in the cleared sample, wherein steps (a) through (g) are performed for a number of times sufficient to bring the concentration of the at least one molecular species of interest in the sample up to the level of detection;
(h) electrophoretically separating the molecular species of interest in the cleared sample; and
(i) detecting the separated molecular species of interest.

26. The method of claim 25 wherein the molecular species of interest in the sample are selected from the group consisting of antibodies, proteins, peptides, peptidomemetics, peptide-nucleic acids, oligonucleotides, aptamers, lipids, polysaccharides, liposaccharides, lipoproteins, glycoproteins and small molecules.

27. The method of claim 25 wherein the acidity of the cleared sample is adjusted to a range from about 5.0 to about 7.0 pH units.

28. The method of claim 25 wherein the optional denaturing step (d) comprises:
(i) adding at least one detergent to the cleared sample; and
(ii) heating the cleared sample in presence of at least one detergent for a period of time from about 1 minute to about 10 minutes to the temperature from about 60° C. to about 100° C.

29. The method of claim 28 wherein heating is performed for about 2 minutes at about 90° C.

30. The method of claim 28 wherein the detergent is sodium dodecyl sulfate.

31. The method of claim 25 wherein step (f) includes introducing a low-electrolyte plug into said separation device, said low-electrolyte plug disposed between the high-electrolyte buffer and the cleared sample at the inlet end the capillary, thereby forming a boundary between the low-electrolyte plug and the high-electrolyte buffer.

32. The method of claim 31 wherein the high-electrolyte buffer and the low-electrolyte plug have substantially different acidities.

33. The method of claim 31 wherein the high electrolyte buffer has an acidity of about 7.0 to about 9.0 pH units and the low-electrolyte plug has an acidity of about 7.0 pH units.

34. The method of claim 25 wherein step (g) comprises:
(i) applying voltage between the inlet end and the outlet end of the capillary for a period of time sufficient to cause electrophoretic migration of molecular species of interest in the cleared sample up to the boundary between the low-electrolyte plug and the high-electrolyte buffer, thereby pre-focusing the cleared sample; and
(ii) applying a pressure differential between the inlet end and the outlet end of the capillary for a period of time sufficient to cause the pre-focused molecular species of interest to be pushed substantially toward the inlet end, thereby concentrating and, optionally, desalting the sample.

35. A method of detecting at least one protein species of interest in a crude sample, comprising:
(a) acquiring a crude sample containing at least one protein species of interest, at least one rough component, larger than the at least one protein species of interest, and at least one fine component smaller than the at least one protein species of interest;
(b) separating the rough component and, optionally, the fine component from the protein species of interest, thereby producing a cleared sample comprising at least one protein species of interest;
(c) optionally, adjusting the acidity of the cleared sample to between about 5.0 and about 7.0 pH-units;
(d) optionally, at least partially denaturing the at least one protein species in the cleared sample by
(i) adding at least one detergent to the cleared sample; and
(ii) heating the cleared sample in the presence of at least one detergent;
(e) optionally, removing insoluble contaminants contained in the cleared sample;
(f) introducing the cleared sample into a separation device comprising:
(i) a capillary, at least partially filled with a high-electrolyte buffer, having an acidity between about 7.0 and about 9.0 pH units, said capillary having an inlet end and an outlet end;
(ii) a means for applying voltage between the inlet end and the outlet end of said capillary; and
(iii) a means for applying pressure differential between the inlet end and the outlet end of said capillary;
whereby a low-electrolyte aqueous solution plug having the acidity of about 7.0 pH units is introduced into the separation device, said low-electrolyte aqueous plug disposed between the high-electrolyte buffer and the cleared sample at the inlet end the capillary, thereby forming a boundary between the low-electrolyte aqueous plug and the high-electrolyte buffer;
(g) pre-focusing and, optionally, desalting the cleared sample, said pre-focusing and desalting comprising:
(i) applying voltage between the inlet end and the outlet end of the capillary for a period of time sufficient to cause electrophoretic migration of the at least one protein species in the cleared sample up to the boundary between the low-electrolyte aqueous plug and the high-electrolyte buffer, thereby pre-focusing and desalting the cleared sample; and
(ii) applying a pressure differential between the inlet end and the outlet end of the capillary for a period of time sufficient to cause the pre-focused protein species of interest to be pushed substantially toward the inlet end of the capillary;
(h) electrophoretically separating the protein species in the cleared sample, comprising the step of applying voltage between the inlet end and the outlet end of the capillary; and
(i) detecting the separated protein species.

36. The method of claim 35 wherein optional step (d) comprises:
(i) adding sodium dodecyl sulfate to the cleared sample; and
(ii) heating the cleared sample in the presence of sodium dodecyl sulfate for about 2 minutes at about 90° C.

37. The method of claim 35 wherein acidity of the high-electrolyte buffer is about 7.0 to about 9.0 pH units.
